(19) United States
(12) Patent Application Publication
(10) Pub. No.: US 2012/0123131 A1
(43) Pub. Date: May 17, 2012

(54) PROCESS FOR THE PREPARATION OF STRONTIUM RANELATE

(76) Inventors: Koilpillai Joseph Prabahar, Tamilnadu (IN); Pravin Bhalchandra Kulkarni, Kalyan (IN); Prashant Bhaskarrao Patil, Kalyan (IN); Kapil Ramesh Hire, Navi Mumbai (IN)

(21) Appl. No.: 13/059,995

(22) PCT Filed: Aug. 8, 2009
(86) PCT No.: PCT/IN2009/000451

§ 371 Date: Apr. 26, 2011

(30) Foreign Application Priority Data
Aug. 22, 2008 (IN) 1774/MUM/2008

(51) Int. Cl. C07F 3/00; C07D 233/58

(52) U.S. Cl. 548/335.1; 549/61

(57) ABSTRACT

The present invention relates to an improved process for the synthesis of strontium ranelate or hydrates thereof. More particularly, the present invention relates to an effective process for the preparation of a compound of formula III, which is a useful intermediate in the synthesis of strontium ranelate.

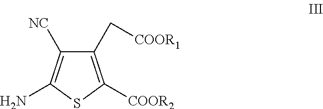

wherein R₁ and R₂ represents substituted or unsubstituted linear or branched C₁-C₆ alkyl group or C₃-C₁₂ cyclic group.

### PRIORITY

This application is a 35 U.S.C. 371 National Stage Filing of International Application No. PCT/IN2009/000451, filed on Aug. 12, 2009, which claims priority under 35 U.S.C. 119 (a-d) to IN 1774/MUM/2008 filed on Aug. 22, 2008, the contents of which is incorporated by reference herein.

### BACKGROUND OF THE INVENTION

1. Technical Field

The present invention relates to an improved process for the synthesis of strontium ranelate or hydrates thereof. More particularly the present invention relates to an effective process for the preparation of a compound of formula III, which is a useful intermediate in the synthesis of strontium ranelate.

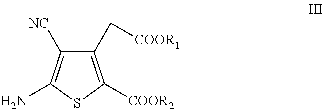

wherein R₁ and R₂ represents substituted or unsubstituted linear or branched C₁-C₆ alkyl group or C₃-C₁₂ cyclic group.

2. Description of the Related Art

Strontium ranelate, the distrontium salt of 5-[bis(carboxymethyl)amino]-3-carboxymethyl-4-cyano-2-thiophenecarboxylic acid is represented by the structure of formula I.

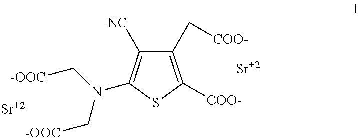

Strontium ranelate has very valuable pharmacological and therapeutic properties, especially pronounced anti-osteoporotic properties, making it useful in the treatment of bone diseases. Commercially, strontium ranelate is available under the trade name Protelos® in Europe.

U.S. Pat. No. 5,128,367 and related European Patent No. 0415850 describes divalent metal salts of 2-[N,N-di(carboxymethyl)amino]-3-cyano-4-carboxymethylthiophene-5-carboxylic acid such as strontium ranelate and its tetrahydrate, heptahydrate and octahydrate. The '367 patent discloses the synthesis of strontium ranelate from the tetraester compound of formula II,

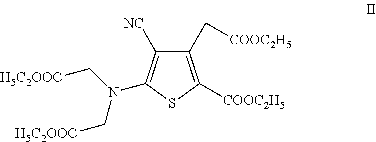

U.S. Pat. No. 7,214,805 and the literature (M. Wierzbicki et al., Bull. Soc. Chim. (1975), pages 1786-1792) disclose the synthesis of the compound of formula II, an intermediate in the preparation of strontium ranelate, comprising alkylation reaction of a compound of formula III (wherein R₁ and R₂ is ethyl) with a compound of formula IV (wherein R′ is ethyl) to give a compound of formula II.

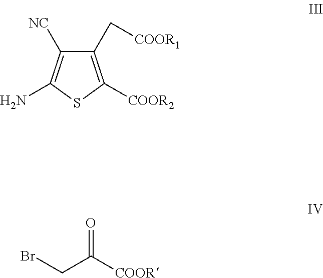

U.S. Pat. No. 7,091,364 discloses the process for the preparation of tetraester compounds, which are intermediates in the preparation of strontium ranelate, including the compound of formula II, from the reaction of a compound of formula III with a compound of formula IV (wherein R′ is linear or branched alkyl) in an organic solvent in the presence of quaternary ammonium compounds at reflux temperature.

U.S. Pat. No. 7,105,683 and the literature (Dinesh W. Rangnekar et al., J. Chem. Tech. Biotechnol. (1990) 47, pages 39-46) disclose the synthesis of the compound of formula III, comprising reaction of 1,3-acetonedicarboxylic acid diethyl ester, malononitrile and sulfur in ethyl alcohol in the presence of bases like morpholine or diethyl amine. The compound of formula III is reported through the formation of an intermediate, an enolate addition salt with bases such as morpholine, diethylamine. The poor to moderate yield of compound of formula III, obtained may be ascribed to the poor stability of these enolate intermediates formed during the reaction.

There are evolving and more rigorous requirements demanded of drug manufacturers and with the prevailing disadvantages present with the prior art, there is a need for an improved process for the preparation of strontium ranelate and its intermediates, which circumvents the formation of process related impurities, while ensuring a target strontium ranelate product with optimum yield and purity.

Surprisingly, it has been found that the development of stable enolate intermediate compounds of general formula V, and compound of particular formula IX, has significant advantages in getting a compound of formula III with good yield and high purity, providing the final strontium ranelate with high purity for pharmaceutical use.

The present invention provides a cost effective industrial process for the preparation of strontium ranelate or hydrate thereof.

### SUMMARY OF THE INVENTION

The present invention relates generally to an improved process for the preparation of a strontium ranelate of formula I or hydrate thereof. More particularly to compounds and processes for their preparation, whereupon said compounds are intermediates useful in the preparation of a strontium ranelate of formula I or hydrate thereof.

The present invention provides strontium ranelate or hydrate thereof having less than about 0.15 area % of 5-[bis(carboxymethyl)amino]-4-cyano-2-(methoxycarbonyl)-3-thiopheneacetic acid methyl ester (Impurity A), as measured by HPLC.

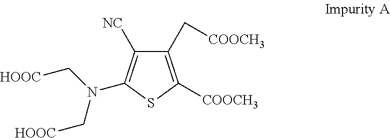

The present invention further provides strontium ranelate or hydrate thereof, having less than about 0.02 area % of Impurity A, as measured by HPLC.

The present invention further provides strontium ranelate of formula I or hydrate thereof, prepared by the processes herein described, having a purity of at least about 99.5 area % as measured by HPLC.

The present invention provides a 5-amino-4-cyano-2-(methoxycarbonyl)-3-thiopheneacetic acid methyl ester, a compound of formula III, (wherein R₁═R₂=methyl)

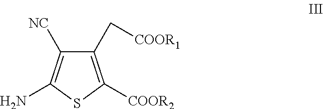

having a purity of more than about 99.7%, as measured by HPLC.

The present invention provides a 3-(dicyanomethylene)-5-hydroxy-5-methoxy-4-pentenoic acid methyl ester compound with imidazole, a compound of formula IX

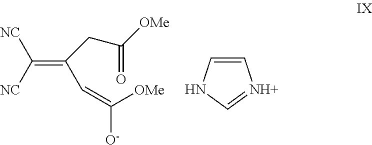

The present invention provides a 3-(dicyanomethylene)-5-hydroxy-5-methoxy-4-pentenoic acid methyl ester compound with imidazole, a compound of formula IX, characterized by an IR spectrum, which is substantially in accordance with FIG. 1.

The present invention further provides a 3-(dicyanomethylene)-5-hydroxy-5-methoxy-4-pentenoic acid methyl ester compound with imidazole, a compound of formula IX, characterized by a ¹H-NMR spectrum, which is substantially in accordance with FIG. 2.

The present invention further provides a compound of formula IX, having a purity of at least about 99.9 area % as measured by HPLC.

The present invention provides a process for the preparation of strontium ranelate of formula I, or hydrate thereof,

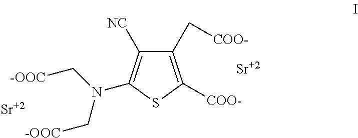

which is defined in Scheme 1
wherein R₁, R₂, R₆ and R₇ represents substituted or unsubstituted linear or branched C₁-C₆ alkyl group or C₃-C₁₂ cyclic group; R₃, R₄ and R₅ independently represents hydrogen or substituted or unsubstituted linear or branched C₁-C₆ alkyl group; R′ represents linear or branched C₁-C₆ alkyl group,
comprising a) reacting a compound of formulae (VI and VII) with imidazole of formula VIII to produce a compound of general formula V; b) reacting the compound of formula V with sulfur effectuating to a compound of formula III; c) reacting the compound of formula III with a compound of formula IV to form a compound of formula IIa; d) converting the compound of formula IIa to its corresponding strontium salt.

The present invention provides a process for the preparation of 5-amino-4-cyano-2-(methoxycarbonyl)-3-thiopheneacetic acid methyl ester, the compound of formula III,

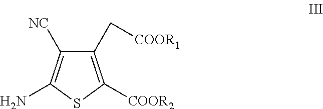

wherein R₁ and R₂ represents substituted or unsubstituted linear or branched C₁-C₆ alkyl group or C₃-C₁₂ cyclic group, comprising:
a) reacting a compound of formula VI and a compound of formula VII,

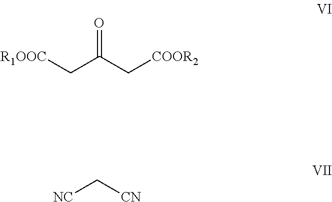

wherein R₁ and R₂ are as described above,
with an imidazole compound of formula VIII,

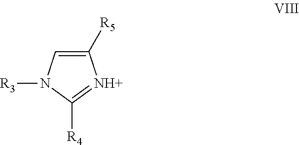

wherein R₃, R₄ and R₅ independently represents hydrogen or substituted or unsubstituted linear or branched C₁-C₆ alkyl group,
in the presence of an organic solvent to form a compound of formula V,

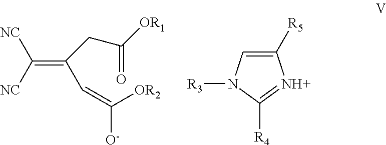

wherein R₁ and R₂ and R₃, R₄ and R₅ are as described previously,
b) reacting the compound of formula V with sulfur to provide a compound of formula III.

The present invention provides a process for the preparation of the compound of formula V,

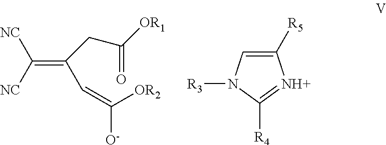

comprising reacting a compound of formula VI and a compound of formula VII,

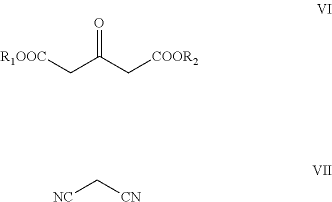

wherein R₁ and R₂ are as described above,
with an imidazole compound of formula VIII,

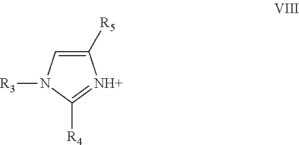

wherein R₃, R₄ and R₅ are as described above,
in the presence of an organic solvent to form a compound of formula V.

The present invention provides a process for the preparation of 3-(dicyanomethylene)-5-hydroxy-5-methoxy-4-pentenoic acid methyl ester compound with imidazole, a compound of formula IX,

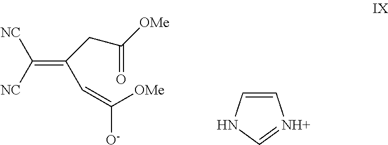

comprising reacting a compound of formula VI and a compound of formula VII,

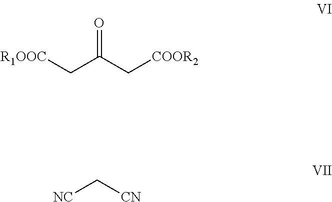

wherein R₁ and R₂ both represents methyl group,
with an imidazole, in the presence of an organic solvent to form a compound of formula IX.

The present invention provides a pharmaceutical composition comprising strontium ranelate or hydrate obtained by the processes herein described, having purity greater than about 99.0 area % as measured by HPLC and at least a pharmaceutically acceptable carrier.

### BRIEF DESCRIPTION OF THE DRAWING

FIG. 1: is an Infrared (IR) spectrum of an imidazole adduct obtained according to Example 1.

FIG. 2: is a proton Nuclear Magnetic Resonance (¹H-NMR) spectrum of an imidazole adduct obtained according to Example 1.

### DETAILED DESCRIPTION OF THE INVENTION

As mentioned above, the present invention is directed to an improved process for the preparation of strontium ranelate or hydrate thereof.

Present health care reforms and legislation lead to evolving and increasingly rigorous requirements demanded of drug manufacturers. Subsequent therefrom and coupled with prevailing disadvantages, which may be present with the prior art processes, paves opportunities for improved processes for the preparation of strontium ranelate and its intermediates, which would circumvent the formation of process related impurities, while ensuring a target strontium ranelate product with optimum yield and purity.

Surprisingly, it has been found that the development of stable enolate intermediate compounds of general formula V, and a compound of particular formula IX, has a significant advantage in getting a compound of formula III with a good yield and high purity, providing the final strontium ranelate with high purity for pharmaceutical use.

The present invention provides a cost effective industrial process for the preparation of strontium ranelate or hydrate thereof.

In an embodiment, the present invention provides strontium ranelate or hydrate thereof, having less than about 0.15 area % of 5-[bis(carboxymethyl)amino]-4-cyano-2-(methoxycarbonyl)-3-thiopheneacetic acid methyl ester (Impurity A), as measured by HPLC.

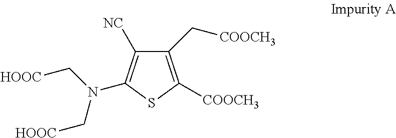

In yet another embodiment, the present invention provides strontium ranelate or hydrate thereof, having less than about 0.02 area % of impurity A, as measured by HPLC.

In still another embodiment, the present invention provides strontium ranelate or hydrate thereof having impurity A, having less than about 0.002% (below detection limit) as measured by HPLC.

In yet another embodiment, the present invention provides strontium ranelate or hydrate thereof, having less than about 0.001 area % (below detection limit) of 5-[bis(2-ethoxy-2-oxoethoxy)amino]-4-cyano-2-(methoxycarbonyl)-3-thiopheneacetic acid methyl ester (Impurity B), as measured by HPLC.

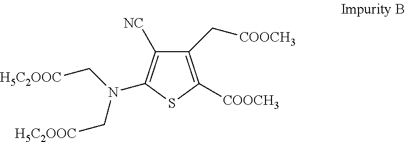

In an embodiment, the present invention provides strontium ranelate of formula I or hydrate thereof, obtained by the processes herein described, having purity greater than about 97.0% to about 99.9%, preferably greater than about 99.0% to about 99.8%, more preferably greater than about 99.5% to about 99.8% as measured by HPLC.

In yet another embodiment, the present invention provides strontium ranelate of formula I or hydrate thereof, obtained by the processes herein described, having individual impurities lower than about 1.0%, preferably lower than about 0.5%, more preferably lower than about 0.15% as measured by area under HPLC peaks.

In another embodiment, the present invention provides a 5-amino-4-cyano-2-(methoxycarbonyl)-3-thiopheneacetic acid methyl ester, a compound of formula III (wherein R₁ and R₂ are methyl)

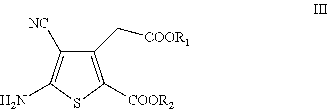

In yet another embodiment, the present invention provides a compound of formula V,

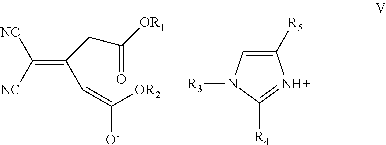

wherein R₁ and R₂ and R₃, R₄ and R₅ are as described above,

In yet another embodiment, the present invention provides a 3-(dicyanomethylene)-5-hydroxy-5-methoxy-4-pentenoic acid methyl ester compound with imidazole, a compound of formula IX.

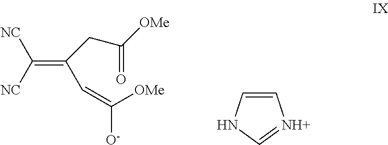

In yet another embodiment, the present invention provides a compound of formula IX, having a purity of more than about 99 area %, as measured by HPLC.

In yet another embodiment, the present invention provides a compound of formula IX, having a purity of at least about 99.9 area %, as measured by HPLC.

In yet another embodiment, the present invention provides a process for preparing strontium ranelate of formula I or hydrate thereof, as shown in Scheme 1

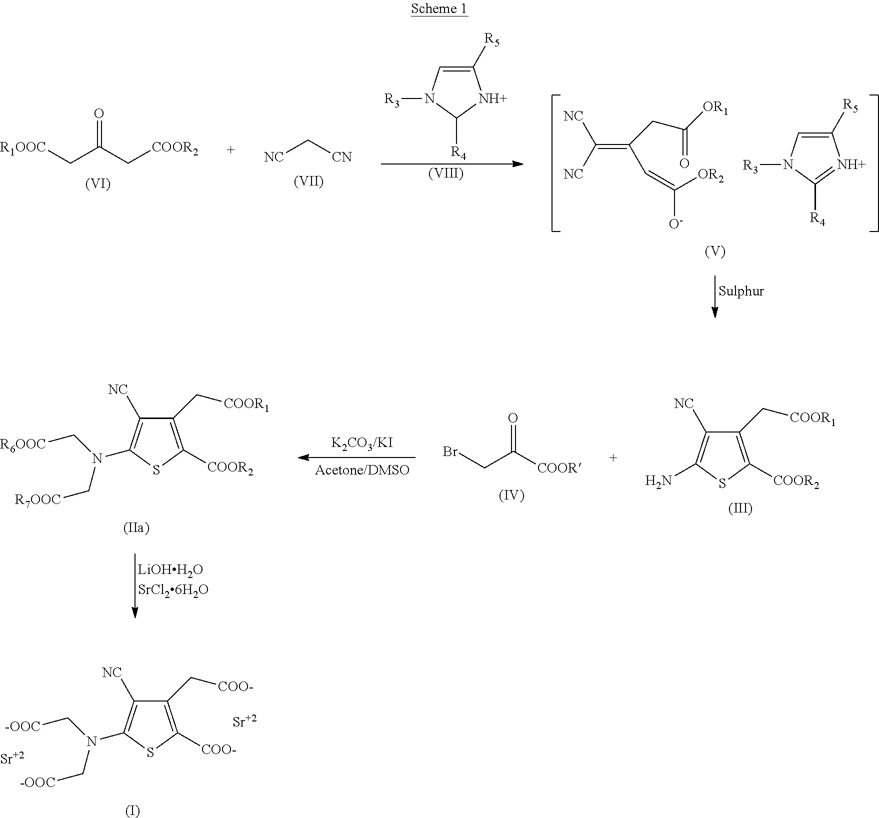

wherein R₁, R₂, R₆ and R₇ represents substituted or unsubstituted linear or branched C₁-C₆ alkyl group or C₃-C₁₂ cyclic group; R₃, R₄ and R₅ independently represents hydrogen or substituted or unsubstituted linear or branched C₁-C₆ alkyl group; R′ represents linear or branched C₁-C₆ alkyl group, comprising:
a) reacting compound of formula III,

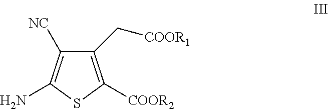

wherein R₁ and R₂ are as described above,
with a compound of formula IV,

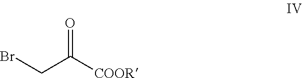

wherein R′ is as described above,
in the presence of catalytic amount of potassium iodide and potassium carbonate in an organic solvent or mixture of organic solvent, to form a compound of formula IIa.

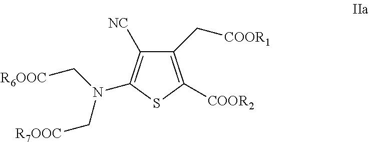

wherein R₁, R₂ and R₆, R₇ are as described above,
b) reacting a compound of formula IIa, with an inorganic acid salt of strontium in the presence of an organic solvent and lithium base.

Organic solvent used in step a) includes acetone, dimethylsulfoxide, acetonitrile, tetrahydrofuran (THF), 2-methyl tetrahydrofuran, N-methylpyrrolidone, C₁-C₄ alcoholic solvents such as methanol, ethanol, isopropanol, isobutanol, water, and the like or mixtures thereof. Preferably, acetone, dimethylsulfoxide and isopropanol.

Suitable inorganic acid salts of strontium used in step b) includes, but are not limited to strontium chloride, strontium chloride hexahydrate, strontium nitrate, strontium bromide, strontium sulfate and the like and mixtures thereof. Preferably, strontium chloride hexahydrate.

Suitable organic solvent used in step b) includes, but are not limited to alcohols, cyclic ethers, water, ketones, nitriles, and the like or mixture thereof. Suitable alcoholic solvent includes C₁-C₅ alcohols such as methanol, ethanol, propanol, isopropanol and the like; cyclic ethers include tetrahydrofuran, dioxane and the like; ketones include C₁-C₁₀ ketone such as acetone, methyl ethyl ketone, diethyl ketone, methyl isopropyl ketone, methyl isobutyl ketone and the like; suitable nitriles include, but are not limited to, acetonitrile and the like or mixture thereof. Preferably, tetrahydrofuran.

Lithium base used in step b) includes, but is not limited to, lithium hydroxide, lithium carbonate, lithium hydroxide monohydrate and the like and mixtures thereof. Preferably, lithium hydroxide monohydrate.

The reaction of the compound of formula III and IV can be carried out at room temperature and to this a mixture of potassium iodide and potassium carbonate is added. The obtained reaction mass is stirred at about 20° C. to about 60° C. for about 5 hours to about 10 hours, preferably at about 35° C. to about 40° C. for about 6 hours to about 8 hours. The amount of compound IV is from about 1 mole to about 5 moles equivalent with respect to the compound of formula III, preferably the amount of compound IV is about 2.2 moles per mole compound of formula III. The amount of potassium carbonate is from about 2 moles to about 5 moles equivalent with respect to the compound of formula III, preferably the amount of potassium carbonate is about 2.5 moles per mole compound of formula III. The amount of potassium iodide is from about 2% w/w to about 10% w/w equivalent with respect to the compound of formula III, preferably the amount of potassium iodide is about 5% w/w per mole compound of formula III.

The compound of formula IIa is reacted with aqueous lithium hydroxide and aqueous strontium chloride at about 0° C. to about 10° C., preferably at about 0° C. to about 5° C. The obtained reaction mixture is stirred at about 10 hours to about 30 hours, preferably at about 15 hours to about 20 hours at room temperature. The amount of lithium base is from about 2 moles to about 10 moles equivalent with respect to the compound of formula IIa, preferably the amount of lithium base is about 4.5 moles per mole compound of formula IIa. The amount of inorganic acid salt of strontium is from about 2 moles to about 6 moles equivalent with respect to the compound of formula IIa, preferably the amount of inorganic acid salt of strontium is about 2.25 moles per mole compound of formula IIa. After completion of the reaction the reaction mass is filtered and wash with water. The obtained resulting material dried at about 30° C. to about 35° C. under reduces pressure to provide strontium ranelate octahydrate.

After completion of the reaction, the desired compounds can be obtained from the reaction mixture by conventional means known in the art. For example, the working-up of reaction mixtures, especially in order to isolate desired compounds, follows customary procedures, known to the organic chemists skilled in the norms of the art and steps, e.g. selected from the group comprising but not limited to extraction, neutralization, crystallization, chromatography, evaporation, drying, filtration, centrifugation and the like. Preferably, crystallization.

In one embodiment, the present invention provides a process for the preparation of 5-amino-4-cyano-2-(methoxycarbonyl)-3-thiopheneacetic acid methyl ester, the compound of formula III,

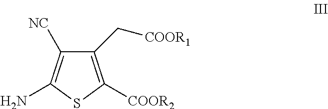

wherein R₁ and R₂ represents substituted or unsubstituted linear or branched C₁-C₆ alkyl group or C₃-C₁₂ cyclic group, comprising:
a) reacting a compound of formula VI and a compound of formula VII,

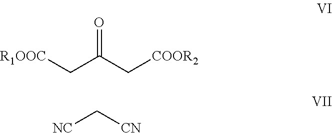

wherein R₁ and R₂ are as described above,
with an imidazole compound of formula VIII,

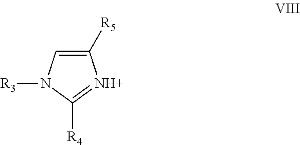

wherein R₃, R₄ and R₅ independently represents hydrogen or substituted or unsubstituted linear or branched C₁-C₆ alkyl group,
in the presence of an organic solvent to form a compound of formula V,

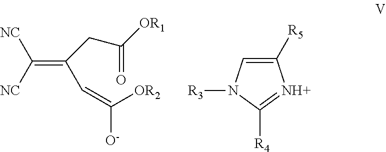

wherein R₁ and R₂ and R₃, R₄ and R₅ are as described above,
b) reacting the compound of formula V with sulfur to provide a compound of formula III.

The C₁-C₆ alkyl group may be substituted or unsubstituted linear or branched and is, for example methyl, ethyl, propyl, isopropyl, butyl, isobutyl, secondary butyl or tertiary butyl, pentyl, hexyl and the like. Preferably, methyl.

The C₃-C₁₂ cyclic group may be substituted or unsubstituted and is, for example, cyclopropyl, cyclobutyl, cyclopentyl, cyclohexyl, cycloheptyl, cyclooctyl, cyclodecyl and their halo, nitro, amino derivatives and the like. Preferably, cyclopropyl.

The organic solvent used is selected from a C₁-C₄ alcohol, such as methanol, ethanol, n-propanol, isopropanol, n-butanol, isobutanol, tert-butanol, ketones such an acetone, methyl isopropyl ketone, methyl isobutyl ketone and the like, nitriles such as acetonitrile and the like or water or mixtures thereof. Preferably, methanol and isopropanol.

The reaction of the compound of formula VI and VII can be carried out at room temperature and reaction mixture obtained is stirred at about 20° C. to about 80° C. for about 1 hour to about 8 hours, preferably at about 25° C. to about 65° C. for about 1 hour to about 4 hours. To the obtained reaction mixture (i.e. VI and VII) imidazole compound is added at room temperature. The amount of imidazole compound is from about 1 mole to about 3 moles equivalent with respect to the compound of formula VII, preferably the amount of imidazole compound is 1 mole per mole compound of formula VII.

The stable enolate intermediate compound of formula V, which can, if desired, be isolated. The reaction can be worked up conventionally by the concentration of the reaction mass and compound of formula V can be isolated by trituration with a solvent like isopropyl alcohol, methyl isobutyl ketone and the like, which further reacts with sulfur at reflux temperature for about 8 hours to about 16 hours, preferably for about 10 hours to about 12 hours.

In yet another embodiment, the present invention provides a process for preparing a compound of formula V, comprising reacting a compound of formula VI and a compound of formula VII,

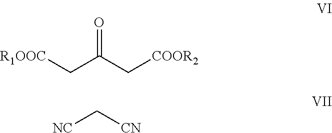

wherein R₁ and R₂ are as described above,
with an imidazole compound of formula VIII,

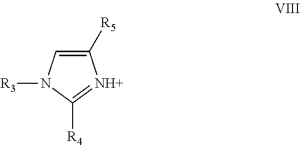

wherein R₃, R₄ and R₅ are as described above,
in the presence of an organic solvent to form a compound of formula V.

In yet another embodiment, the present invention provides a process for the preparation of 3-(dicyanomethylene)-5-hydroxy-5-methoxy-4-pentenoic acid methyl ester compound with imidazole, a compound of formula IX,

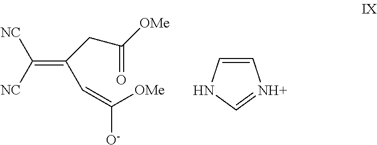

comprising reacting a compound of formula VI and a compound of formula VII,

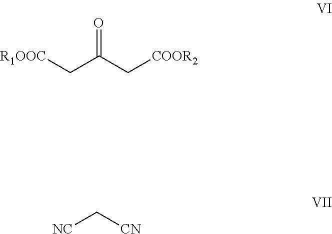

wherein both R₁ and R₂ represent a methyl group,
with an imidazole, in the presence of an organic solvent to form a compound of formula IX.

Without being bound by any theory, it is believed that as the positive charge of the imidazole moiety is delocalized, due to resonance, thus the stability of enolate intermediate compound of formula IX addition salt is enhanced. This stable enolate intermediate compound of formula IX can be isolated or used in-situ, which further reacts with sulfur to provide compound of formula III (wherein R₁ and R₂ is methyl).

In yet another embodiment, strontium ranelate obtained by the processes herein described, has residual organic solvents or organic volatile impurities comprises less than the amount recommended for pharmaceutical products, as set forth for example in ICH guidelines and U.S. pharmacopoeia; acetone (ICH Limit: 5000 ppm) is less than about 22 ppm (below detection limit), dimethylsulfoxide (ICH Limit: 5000 ppm) is less than about 60 ppm (below detection limit), ethyl acetate (ICH Limit: 5000 ppm) is less than about 11 ppm (below detection limit), isopropyl alcohol (ICH Limit: 5000 ppm) is less than about 19 ppm (below detection limit), cyclohexane (ICH Limit: 3880 ppm) is less than about 13 ppm (below detection limit), methanol (ICH Limit: 3000 ppm) is less than about 10 ppm (below detection limit), tetrahydrofuran (THF) (ICH Limit: 720 ppm) is less than about 2 ppm (below detection limit).

In yet another embodiment, the present invention provides pharmaceutical compositions comprising strontium ranelate or hydrate thereof obtained by the processes herein described, having a D₅₀ and D₉₀ particle size of less than about 150 microns, preferably less than about 100 microns, more preferably less than about 50 microns, still more preferably less than about 20 microns, still more preferably less than about 15 microns and most preferably less than about 10 microns. The particle size disclosed here can be obtained by, for example, any milling, grinding, micronizing or other particle size reduction method known in the art to bring the solid state strontium ranelate into any of the foregoing desired particle size range.

The strontium ranelate or hydrate thereof disclosed herein for use in the pharmaceutical compositions of the present invention is particularly useful in the treatment of a bone disease or condition such as, for example, osteoporosis, osteoarthritis, osteopetrosis, osteopenia and Paget's disease, hypercalcemia of malignancy, periarticular erosions in rheumatoid arthritis, osteodystrophy, myositis ossificans, Bechterew's disease, malignant hypercalcemia, osteolytic lesions produced by bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, and for the improvement of fracture healing after traumatic or atraumatic fracture

The processes, herein described, for the preparation of strontium ranelate and intermediates of strontium ranelate are simple, eco-friendly, inexpensive, reproducible, robust and well suited on industrial scale.

While the present invention has been described in terms of its specific embodiments, certain modifications and equivalents will be apparent to those skilled in the art and are intended to be included within the scope of the present invention.

### EXAMPLES

For Comparative Examples 1 & 2 are synthesis of intermediate compound of formula III (i.e. 5-amino-4-cyano-2-(methoxycarbonyl)-3-thiopheneacetic acid methyl ester), (according to U.S. Pat. No. 7,105,683 and J. Chem. Tech. Biotechnol. (1990) 47, pages 39-46)

### Comparative Example 1

### Preparation of 5-amino-4-cyano-2-(methoxycarbonyl)-3-thiopheneacetic acid methyl ester (according to U.S. Pat. No. 7,105,683)

A mixture of malononitrile (15.5 g) and morpholine (20.4 g) was added to a solution of 1,3-acetone dicarboxylic acid dimethyl ester (50 g) in methanol (860 ml) at room temperature. This reaction mixture was stirred at 40-45° C. for about 1 to 2 hours. Thereafter, sulfur (7.5 g) was added and the reaction mass was heated to reflux for about 10-12 hours. The reaction mass was cooled and filtered and the filtrate was concentrated under reduced pressure. Water was added to the residue and the precipitated solid was isolated by filtration. The solid was further recrystallized from isopropyl alcohol to get 45 g (61%) of 5-amino-4-cyano-2-(methoxycarbonyl)-3-thiopheneacetic acid methyl ester.

### Comparative Example 2

### Preparation of 5-amino-4-cyano-2-(methoxycarbonyl)-3-thiopheneacetic acid methyl ester (according to J. Chem. Tech. Biotechnol. (1990) 47, 39-46)

A mixture of malononitrile (18 g) and N,N-diethylamine (20.2 g) was added to a solution of 1,3-acetone dicarboxylic acid dimethyl ester (50 g) in methanol (700 ml) at room temperature. This reaction mixture was stirred at 40-45° C. for about 1 to about 2 hours. Thereafter, sulfur (8.75 g) was added and the reaction mass was heated to reflux for about 10-12 hours. The reaction mass was cooled and filtered and the filtrate was concentrated under reduced pressure. Water was added to the residue and the precipitated solid was isolated by filtration. The solid was further recrystallized from isopropyl alcohol to get 38 g (52%) of 5-amino-4-cyano-2-(methoxycarbonyl)-3-thiopheneacetic acid methyl ester.

### Example 1

### Preparation of Compound IX: 3-(dicyanomethylene)-5-hydroxy-5-methoxy-4-pentenoic acid methyl ester compound with imidazole (1:1)

A mixture of malononitrile (3.6 g) and imidazole (3.7 g) was added to a solution of 1,3-acetone dicarboxylic acid dimethyl ester (10 g) in methanol (150 ml) at room temperature. This reaction mixture was stirred at about 40-45° C. for about 1 hour. Thereafter the reaction mass was concentrated under reduced pressure. The residue mass was further triturated with methyl isobutyl ketone to get 6 g of compound IX, having a purity 99.65% as determined by HPLC.

IR (KBr) cm⁻¹: 2166.55 & 2193.89 (2×CN), 1727.89 (C═O), 1157.2 (—O—CH₃) and ¹H-NMR (proton NMR) having the values as follows: ¹H-NMR (DMSO-d₆), δ (ppm): 3.41 (s, 2H, —CH₂—), 3.56 and 3.70 (2 s, 6H, 2×—OCH₃), 5.04 (s, 1H, ═CH—), 7.68 and 9.06 (2 s, 3H, imidazole hydrogens)

### Example 2

### Preparation of 5-amino-4-cyano-2-(methoxycarbonyl)-3-thiophene acetic acid methyl ester

A mixture of malononitrile (54.1 g) and imidazole (58.48 g) was added to a solution of 1,3-acetone dicarboxylic acid dimethyl ester (150 g) in methanol (2000 ml) at room temperature. This reaction mixture was stirred at 40-45° C. for about 1 to 2 hours. Thereafter, sulfur (26.25 g) was added and the reaction mass was heated to reflux for about 10-12 hours. The reaction mass was cooled and filtered and the filtrate was concentrated under reduced pressure. Water was added to the residue and the precipitated solid was isolated by filtration. The solid was further recrystallized from isopropyl alcohol (750 ml) to get 161 g of 5-amino-4-cyano-2-(methoxycarbonyl)-3-thiopheneacetic acid methyl ester.

Three batches were prepared, in the manner described above, the results of which are as follows:

| Batch No. | Purity by HPLC | Yield |
|---|---|---|
| 1 | 99.78% | 73.52% |
| 2 | 99.74% | 73.52% |
| 3 | 99.74% | 73.98% |

### Example 3

### Preparation of 5-[bis(2-ethoxy-2-oxoethyl)amino]-4-cyano-2-(methoxycarbonyl)-3-thiopheneacetic acid methyl ester

5-amino-4-cyano-2-(methoxycarbonyl)-3-thiopheneacetic acid methyl ester (50 g) was dissolved in a mixture of acetonitrile (200 ml) and dimethylsulfoxide (20 ml) at room temperature. To this solution, a mixture of potassium iodide (2.5 g) and potassium carbonate (68 g) was added. Thereafter, ethyl bromoacetate (75 g) was added to the reaction mass and reaction mass was stirred at about 35-40° C. for about 7 hours. The reaction mass was then cooled and filtered. The filtrate was concentrated and triturated with isopropyl alcohol. The resulting solid was filtered and dried to get 60 g of 5-[bis(2-ethoxy-2-oxoethyl)amino]-4-cyano-2-(methoxycarbonyl)-3-thiopheneacetic acid methyl ester having a purity of 99.74% as determined by HPLC.

### Example 4

### Preparation of 5-[bis(2-ethoxy-2-oxoethyl)amino]-4-cyano-2-(methoxycarbonyl)-3-thiopheneacetic acid methyl ester

5-amino-4-cyano-2-(methoxycarbonyl)-3-thiopheneacetic acid methyl ester (50 g) was dissolved in a mixture of acetone (200 ml) and dimethylsulfoxide (20 ml) at room temperature. To this solution, a mixture of potassium iodide (2.5 g) and potassium carbonate (68 g) was added. Thereafter, ethyl bromoacetate (75 g) was added to the reaction mass and reaction mass was stirred at about 35-40° C. for about 7 hours. The reaction mass was then cooled and filtered. The filtrate was concentrated and triturated with a mixture of isopropyl alcohol and water. The resulting solid was filtered, crystallized from isopropyl alcohol and dried to get 60 g of 5-[bis(2-ethoxy-2-oxoethyl)amino]-4-cyano-2-(methoxycarbonyl)-3-thiopheneacetic acid methyl ester having purity of 99.74% as determined by HPLC.

### Example 5

### Preparation of 5-[bis(carboxymethyl)amino]-2-carboxy-4-cyano-3-thiopheneacetic acid distrontium salt

A mixture of 5-[bis(2-ethoxy-2-oxoethyl)amino]-4-cyano-2-(methoxycarbonyl)-3-thiopheneacetic acid methyl ester (50 g) and tetrahydrofuran (250 ml) was cooled to about 0-5° C. To this mixture, 10% w/v aqueous lithium hydroxide solution (200 ml) was added and the reaction mass was stirred at about 0-5° C. for about 8 hours. After completion of the reaction, a solution of strontium chloride (71 g) in water (300 ml) was added at about 0-5° C. This reaction mixture was stirred for about 15 to 20 hours at room temperature. The precipitated solid, distrontium salt of 5-[bis(2-ethoxy-2-oxoethyl)amino]-4-cyano-2-(methoxycarbonyl)-3-thiopheneacetic acid was filtered off and washed with water. The resulting wet material was dried at about 30-35° C. under reduced pressure to yield 66 g of strontium ranelate octahydrate. Residual solvents (by GC)-acetone: 22 ppm; dimethylsulfoxide: 60 ppm; ethyl acetate: 11 ppm; isopropyl alcohol: 19 ppm; cyclohexane: 13 ppm; methanol: 10 ppm; THF: 2 ppm.

Below table depicts preparation of pure strontium ranelate batches prepared by following the procedure of examples 1 to 5.

| Batch | | | |
|---|---|---|---|
| No. | Purity by HPLC | Impurity A by HPLC | Impurity B by HPLC |
| 1 | 99.85% | 0.002% | 0.001% |
| 2 | 99.73% | 0.002% | 0.001% |
| 3 | 99.71% | 0.02% | 0.001% |

1. Strontium ranelate or hydrate thereof having less than about 0.15 area % of 5-[bis(carboxymethyl)amino]-4-cyano-2-(methoxycarbonyl)-3-thiopheneacetic acid methyl ester (Impurity A), as measured by HPLC.

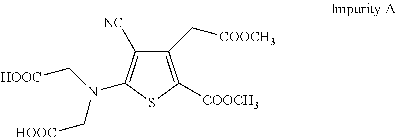

2.-5. (canceled)

6. 3-(dicyanomethylene)-5-hydroxy-5-methoxy-4-pentenoic acid methyl ester compound with imidazole, a compound of formula IX.

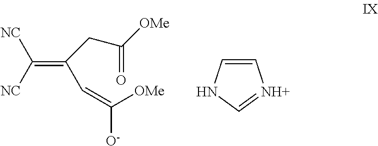

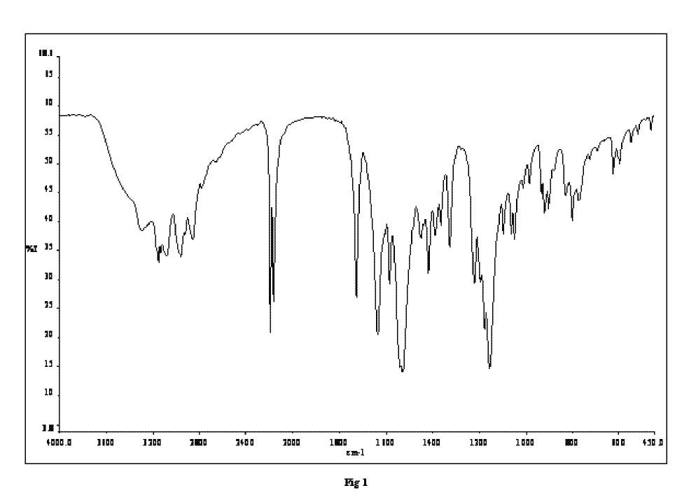

7. 3-(dicyanomethylene)-5-hydroxy-5-methoxy-4-pentenoic acid methyl ester compound with imidazole, a compound of formula IX of claim 6, characterized by an IR spectrum, which is substantially in accordance with FIG. 1.

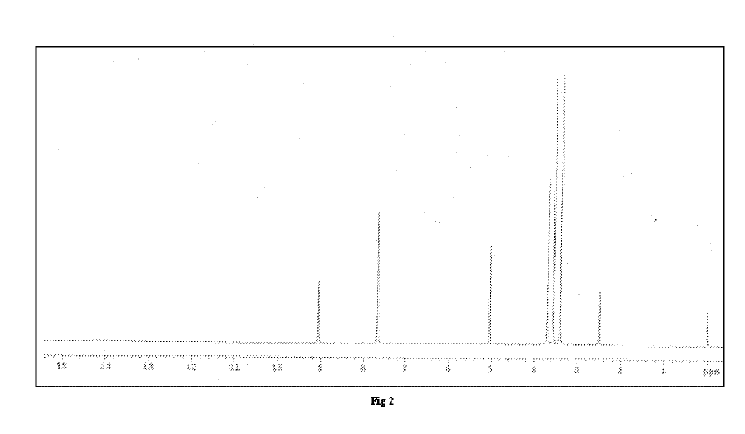

8. 3-(dicyanomethylene)-5-hydroxy-5-methoxy-4-pentenoic acid methyl ester compound with imidazole, a compound of formula IX of claim 6, characterized by a ¹H-NMR spectrum, which is substantially in accordance with FIG. 2.

9. A process, which is defined in Scheme 1, for the preparation of strontium ranelate of formula I, or hydrate thereof, 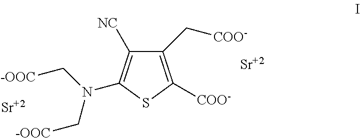 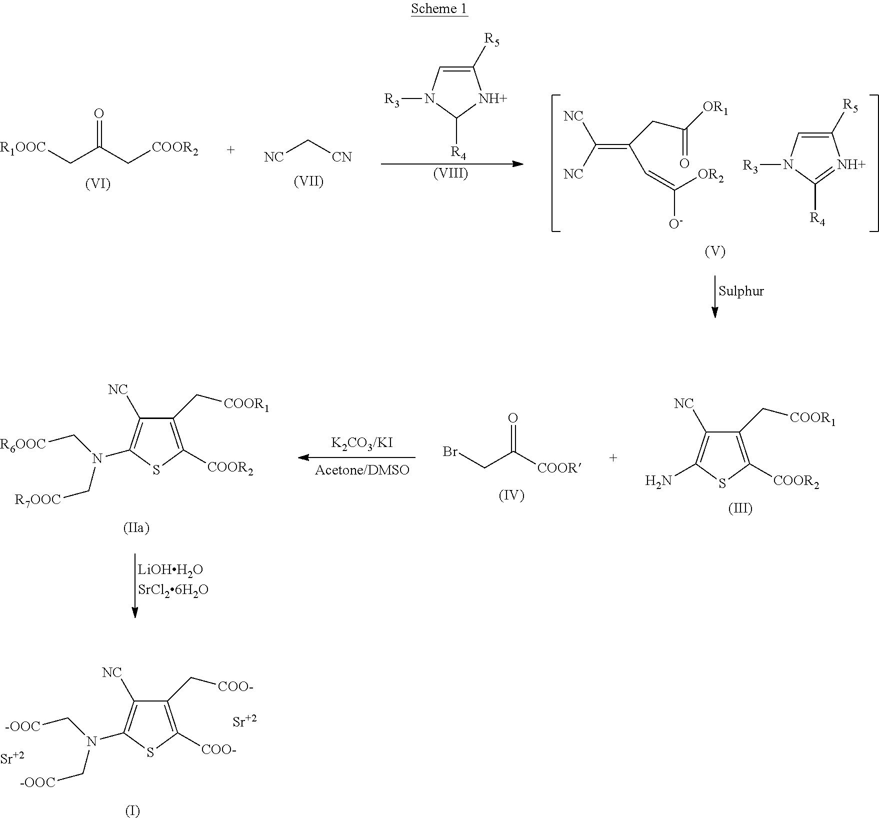
wherein R₁, R₂, R₆ and R₇ represents substituted or unsubstituted linear or branched C₁-C₆ alkyl group or C₃-C₁₂ cyclic group; R₃, R₄ and R₅ independently represents hydrogen or substituted or unsubstituted linear or branched C₁-C₆ alkyl group; R′ represents linear or branched C₁-C₆ alkyl group, comprising:
a) reacting a compound of formulae (VI and VII) with imidazole of formula VIII to produce a compound of general formula V;
b) reacting the compound of formula V with sulfur effectuating to a compound of formula III;
c) reacting the compound of formula III with a compound of formula IV to form a compound of formula IIa;
d) converting the compound of formula IIa to its corresponding strontium salt.

10.-20. (canceled)

21. The process of claim 9, wherein strontium ranelate is in the form of an octahydrate.

22.-24. (canceled)

25. The process of claim 9, wherein the organic solvent is selected from a C₁-C₄ alcohol.

26. The process of claim 9, wherein the temperature of the reaction is from about 20° C. to about 70° C.

27. The process of claim 9, wherein the compound of formula V 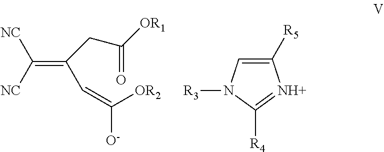
is prepared by a process, comprising reacting a compound of formula VI and a compound of formula VII, 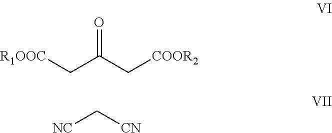
wherein R₁ and R₂ are defined as in claim 22,
with an imidazole compound of formula VIII, 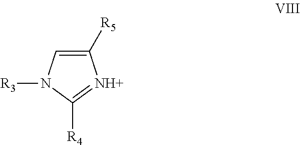
wherein R₃, R₄ and R₅ are defined as in claim 22,
in the presence of an organic solvent to form a compound of formula V.

28. (canceled)

29. A process for the preparation of 3-(dicyanomethylene)-5-hydroxy-5-methoxy-4-pentenoic acid methyl ester compound with imidazole, a compound of formula IX, as defined in claim 6, 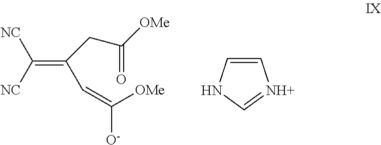
comprising reacting a compound of formula VI and a compound of formula VII, 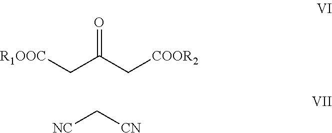
wherein R1 and R2 both represents methyl group,
with an imidazole, in the presence of an organic solvent to form a compound of formula IX.

30. The process of claim 29, wherein the organic solvent is selected from a C₁-C₄ alcohol.
